(19) United States
(12) Patent Application Publication
(10) Pub. No.: US 2008/0214827 A1
(43) Pub. Date: Sep. 4, 2008

(54) Synthesis of Cyanoimino-Benzoimidazoles

(75) Inventors: R. Richard Goehring, Pipersville, PA (US); John Whitehead, Newton, PA (US); Bin Shao, Richboro, PA (US)

Correspondence Address: DAVIDSON DAVIDSON & KAPPEL LLC., 485 SEVENTH AVENUE, 14TH FLOOR, NEW YORK NEW YORK, NY, 10018, US

(73) Assignee: EURO-CELTIQUE S.A., LUXEMBOURG (US)

(21) Appl. No.: 10/585,485

(22) PCT Filed: Feb. 2, 2005
(86) PCT No.: PCT/US05/02824

§ 371 Date: Oct. 17, 2007

Related U.S. Application Data
(60) Provisional application No. 60/541,393, filed on Feb. 3, 2004.

(51) Int. Cl. C07D 401/04

(52) U.S. Cl. 546/273.4

(57) ABSTRACT

Disclosed in certain embodiments is a process for synthesizing a compound of formula (V) and salts thereof.

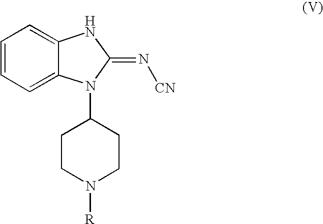

This application claims priority from U.S. Provisional Application No. 60/541,393, filed Feb. 3, 2004, the disclosure of which is hereby incorporated by reference in its entirety.

### BACKGROUND OF THE INVENTION

Chronic pain is a major contributor to disability and is the cause of an untold amount of suffering. The successful treatment of severe and chronic pain is a primary goal of the physician with opioid analgesics being preferred drugs.

Until recently, there was evidence of three major classes of opioid receptors in the central nervous system (CNS), with each class having subtype receptors. These receptor classes were designated as μ, δ and κ. As opiates had a high affinity to these receptors while not being endogenous to the body, research followed in order to identify and isolate the endogenous ligands to these receptors. These ligands were identified as enkephalins, endorphins and dynorphins.

Recent experimentation has led to the identification of a cDNA encoding an opioid receptor-like (ORL1) receptor with a high degree of homology to the known receptor classes. This newly discovered receptor was classified as an opioid receptor based only on structural grounds, as the receptor did not exhibit pharmacological homology. It was initially demonstrated that non-selective ligands having a high affinity for μ, δ and κ receptors had low affinity for the ORL1. This characteristic, along with the fact that an endogenous ligand had not yet been discovered, led to the term “orphan receptor”.

Subsequent research led to the isolation and structure of the endogenous ligand of the ORL1 receptor. This ligand is a seventeen amino acid peptide structurally similar to members of the opioid peptide family.

The discovery of the ORL1 receptor presents an opportunity in drug discovery for novel compounds which can be administered for pain management or other syndromes modulated by this receptor.

WO 02/085357 discloses cyanoimino-benzimidazoles having affinity for the ORL-1 receptor and methods of synthesis thereof.

There exists a need in the art for improved methods of synthesizing cyanoimino-benzimidazoles and for novel compounds thereof.

All documents cited herein, including the foregoing, are incorporated by reference in their entireties for all purposes.

### OBJECTS AND SUMMARY OF THE INVENTION

It is an object of certain embodiments of the present invention to provide novel processes for synthesizing cyanoimino-benzimidazoles.

It is an object of certain embodiments of the present invention to provide novel cyanoimino-benzimidazoles and pharmaceutical compositions thereof.

It is an object of certain embodiments of the present invention to provide novel intermediates useful in the synthesis of cyanoimino-benzimidazoles.

It is an object of certain embodiments of the present invention to provide a process for synthesizing a compound of formula (VI):

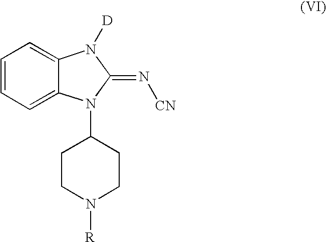

and pharmaceutically acceptable salts thereof, wherein D and R are as disclosed herein.

It is an object of certain embodiments of the present invention to provide a process for synthesizing a compound of formula (V):

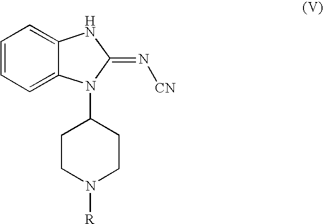

and pharmaceutically acceptable salts thereof, wherein R is as disclosed herein.

It is an object of certain embodiments of the present invention to provide a process for synthesizing a compound of formula (IV):

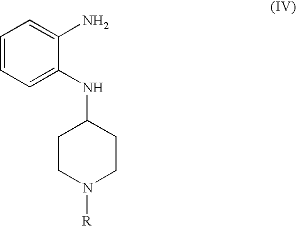

wherein R is as disclosed herein.

It is an object of certain embodiments of the present invention to provide a process for synthesizing a compound of formula (III):

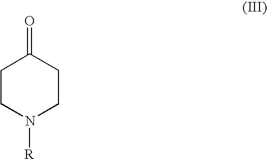

wherein R is as disclosed herein.

It is an object of certain embodiments of the present invention to provide a process for synthesizing a compound of formula (IIIA):

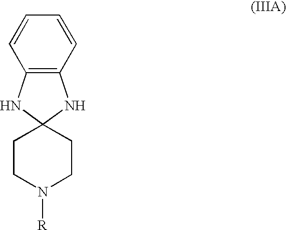

wherein R is as disclosed herein.

It is an object of certain embodiments of the present invention to provide a composition of the formula (VII):

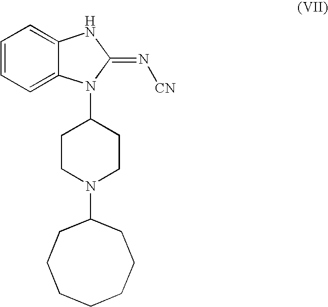

and pharmaceutically acceptable salts thereof.

It is an object of the present invention to provide a method of treating pain in a patient with an effective amount of a compound of formula (VII).

It is an object of the present invention to provide a method of agonizing the ORL1 receptor in a patient with an effective amount of a compound of formula (VII).

It is an object of the present invention to provide compounds of formula (VII) useful as analgesics, anti-inflammatories, diuretics, anesthetics and neuroprotective agents, anti-hypertensives, anti-anxioltics; agents for appetite control; hearing regulators; anti-tussives, anti-asthmatics, modulators of locomotor activity, modulators of learning and memory, regulators of neurotransmitter and hormone release, kidney function modulators, antidepressants, agents to treat memory loss due to Alzheimer's disease or other dementias, anti-epileptics, anti-convulsants, agents to treat withdrawal from alcohol and drugs of addiction, agents to control water balance, agents to control sodium excretion and agents to control arterial blood pressure disorders and methods for administering said compounds.

It is an object of certain embodiments of the present invention to provide a composition of the formula (VII):

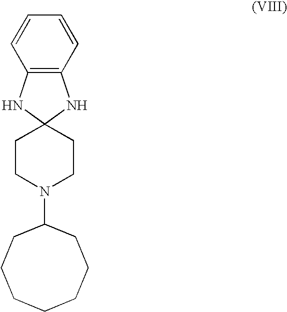

and salts thereof.

In view of the above objects and others, the present invention in certain embodiments is directed to a process for synthesizing a compound of formula (V):

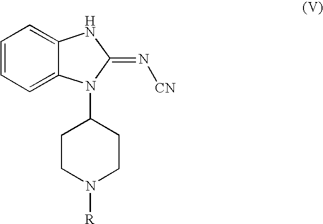

comprising reacting a compound of formula (I)

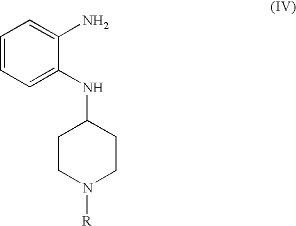

with (A)(A₁)-cyanocarbonimidate to form a compound of formula (V);

wherein A and A₁ are independently selected from methyl, ethyl propyl, phenyl and benzyl; and wherein,

R is Z-R1, wherein

Z is selected from the group consisting of a bond, straight or branched C₁₋₆ alkylene, —NH—, —CH₂O—, —CH₂NH—, —CH₂N(CH₃)—, —NHCH₂—, —CH₂CONH—, —NHCH₂CO—, —CH₂CO—, —COCH₂—, —CH₂COCH₂—, —CH(CH₃)—, —CH═, —O— and —HC═CH—, wherein the carbon and/or nitrogen atoms are unsubstituted or substituted with one or more lower alkyl, hydroxy, halo or alkoxy group;

R₁ is selected from the group consisting of hydrogen, C₁₋₁₀ alkyl, C₃₋₁₂cycloalkyl, C₂₋₁₀alkenyl, amino, C₁₋₁₀alkylamino-, C₃₋₁₂cycloalkylamino-, —COOV₁, —C₁₋₄COOV₁, cyano, cyanoC₁₋₁₀alkyl-, cyanoC₃₋₁₀cycloalkyl-, NH₂SO₂—, NH₂SO₂C₁₋₄alkyl-, NH₂SOC₁₋₄alkyl-, aminocarbonyl-, C₁₋₄alkylaminocarbonyl-, diC₁₋₄alkylaminocarbonyl-, benzyl, C₃₋₁₂ cycloalkenyl-, a monocyclic, bicyclic or tricyclic aryl or heteroaryl ring, a hetero-monocyclic ring, a hetero-bicyclic ring system, and a spiro ring system of the formula (XI):

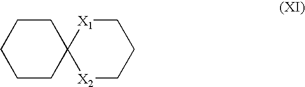

wherein X₁ and X₂ are independently selected from the group consisting of NH, O, S and CH₂; and wherein said alkyl, cycloalkyl, alkenyl, C₁₋₁₀alkylamino-, C₃₋₁₂cycloalkylamino-, or benzyl of R₁ is optionally substituted with 1-3 substituents selected from the group consisting of halogen, hydroxy, C₁₋₁₀ alkyl, C₁₋₁₀ alkoxy, nitro, trifluoromethyl-, cyano, —COOV₁, —C₁₋₄COOV₁, cyanoC₁₋₁₀alkyl-, —C₁₋₅(═O)W₁, —C₁₋₅NHS(═O)₂W₁, —C₁₋₅NHS(═O)W₁, a 5-membered heteroaromaticC₀₋₄alkyl-, phenyl, benzyl, benzyloxy, said phenyl, benzyl, and benzyloxy optionally being substituted with 1-3 substituents selected from the group consisting of halogen, C₁₋₁₀ alkyl-, C₁₋₁₀ alkoxy-, and cyano; and wherein said C₃₋₁₂ cycloalkyl, C₃₋₁₂ cycloalkenyl, monocyclic, bicyclic or tricyclic aryl, heteroaryl ring, hetero-monocyclic ring, hetero-bicyclic ring system, or spiro ring system of the formula (II) is optionally substituted with 1-3 substituents selected from the group consisting of halogen, C₁₋₁₀ alkyl, C₁₋₁₀ alkoxy, nitro, trifluoromethyl-, phenyl, benzyl, phenyloxy and benzyloxy, wherein said phenyl, benzyl, phenyloxy or benzyloxy is optionally substituted with 1-3 substituents selected from the group consisting of halogen, C₁₋₁₀ alkyl, C₁₋₁₀ alkoxy, and cyano;

wherein V₁ is independently selected from H, C₁₋₆ alkyl, C₃₋₆ cycloalkyl, benzyl and phenyl; and

wherein W₁ is hydrogen, C₁₋₁₀ alkyl, C₃₋₁₂ cycloalkyl, C₁₋₁₀ alkoxy, C₃₋₁₂ cycloalkoxy, —CH₂OH, amino, C₁₋₄alkylamino-, diC₁₋₄alkylamino-, or a 5-membered heteroaromatic ring optionally substituted with 1-3 lower alkyl.

In further embodiments, the present invention is directed to a process for synthesizing a compound of formula (IV) by subjecting a compound of formula (III):

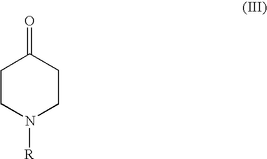

to reductive amination with 1,2-phenylenediamine, an acid and a reducing agent to form a compound of formula (IV), wherein R is as disclosed herein.

In further embodiments, the present invention is directed to a process for synthesizing a compound of formula (IV) by subjecting a compound of formula (III):

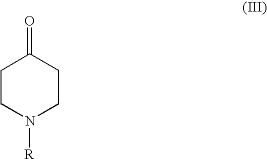

to amination with 1,2-phenylenediamine and an acid to form a compound of formula (IIIA)

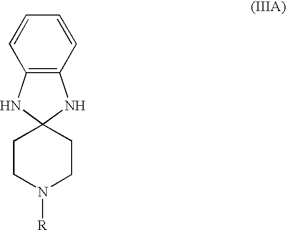

and reducing the compound of (IIIA) with a reducing agent to form a compound of formula (IV), wherein R is as disclosed herein.

In further embodiments, the present invention is directed to a process for preparing a compound of formula (IIIA) from a compound of formula (III) wherein R is as disclosed herein.

In further embodiments, the present invention is directed to a process for preparing a compound of formula (IV) from a compound of formula (IIIA) wherein R is as disclosed herein.

In further embodiments, the present invention is directed to a process for reacting a compound of formula (V) with a D-halogen to form a compound of formula (VI):

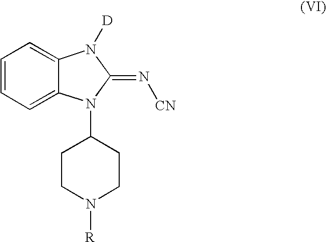

wherein D is selected from the group consisting of C₁₋₁₀ alkyl, C₃₋₁₂ cycloalkyl, C₃₋₁₂ cycloalkylC₁₋₄alkyl-, C₁₋₁₀ alkoxy, C₃₋₁₂ cycloalkoxy-, C₁₋₁₀ alkyl substituted with 1-3 halogen, C₃₋₁₂ cycloalkyl substituted with 1-3 halogen, C₃₋₁₂ cycloalkylC₁₋₄alkyl-substituted with 1-3 halogen, C₁₋₁₀ alkoxy substituted with 1-3 halogen, C₃₋₁₂ cycloalkoxy-substituted with 1-3 halogen, —COOV₁, —C₁₋₄COOV₁, —CH₂OH, —SO₂N(V₁)₂, hydroxyC₁₋₁₀alkyl-, hydroxyC₃₋₁₀cycloalkyl-, cyanoC₁₋₁₀alkyl-, cyanoC₃₋₁₀cycloalkyl-, —CON(V₁)₂, NH₂SO₂C₁₋₄alkyl-, NH₂SOC₁₋₄alkyl-, sulfonylaminoC₁₋₁₀alkyl-, diaminoalkyl-, -sulfonylC₁₋₄alkyl, a 6-membered heterocyclic ring, a 6-membered heteroaromatic ring, a 6-membered heterocyclicC₁₋₄alkyl-, a 6-membered heteroaromaticC₁₋₄alkyl-, a 6-membered aromatic ring, a 6-membered aromaticC₁₋₄ alkyl-, a 5-membered heterocyclic ring optionally substituted with an oxo or thio, a 5-membered heteroaromatic ring, a 5-membered heterocyclicC₁₋₄alkyl-optionally substituted with an oxo or thio, a 5-membered heteroaromaticC₁₋₄alkyl-, —C₁₋₅(═O)W₁—C₁₋₅(═NH)W₁, —C₁₋₅NHC(═O)W₁, —C₁₋₅NHS(═O)₂W₁, —C₁₋₅NHS(═O)W₁, (wherein W₁ is hydrogen, C₁₋₁₀ alkyl, C₃₋₁₂ cycloalkyl, C₁₋₁₀ alkoxy, C₃₋₁₂ cycloalkoxy, —CH₂OH, amino, C₁₋₄alkylamino-, diC₁₋₄alkylamino-) and a 5-membered heteroaromatic ring optionally substituted with 1-3 lower alkyl;

wherein each V₁ is independently selected from H, C₁₋₆ alkyl, C₃₋₆ cycloalkyl, benzyl and phenyl; and

wherein R is as disclosed herein.

In certain embodiments of any of the above formulae, R₁ is an alkyl selected from is methyl, ethyl, propyl, butyl, pentyl, or hexyl.

In certain embodiments of any of the above formulae, R₁ is cycloalkyl selected from cyclohexyl, cycloheptyl, cyclooctyl, cyclononyl, cyclodecyl, or norbornyl.

In certain embodiments of any of the above formulae, R₁ is a bicyclic ring selected from indenyl, quinoline, naphthyl, tetrahydronaphthyl, or decahydronaphthyl.

In certain embodiments of any of the above formulae, R₁ is a tricyclic ring such as dibenzocycloheptyl.

In certain embodiments of any of the above formulae, R₁ is phenyl or benzyl.

In certain embodiments of any of the above formulae, Z is a bond, methyl, or ethyl.

In certain embodiments of any of the above formulae, the Z group is maximally substituted as not to have any hydrogen substitution on the base Z group. For example, if the base Z group is —CH₂—, substitution with two methyl groups would remove hydrogens from the —CH₂— base Z group.

In certain embodiments of any of the above formulae, ZR₁ is cyclohexylethyl-, cyclohexylmethyl-, cyclopentylmethyl-, dimethylcyclohexylmethyl-, phenylethyl-, pyrrolyltrifluoroethyl-, thienyltrifluoroethyl-, pyridylethyl-, cyclopentyl-, cyclohexyl-, cyclooctyl, methoxycyclohexyl-, tetrahydropyranyl-, propylpiperidinyl-, indolylmethyl-, pyrazoylpentyl-, thiazolylethyl-, phenyltrifluoroethyl-, hydroxyhexyl-, methoxyhexyl-, isopropoxybutyl-, hexyl-, or oxocanylpropyl-.

In certain embodiments of any of the above formulae, ZR₁—CH₂COOV₁, tetrazolylmethyl-, cyanomethyl-, NH₂SO₂methyl-, NH₂SOmethyl-, aminocarbonylmethyl-, C₁₋₄alkylaminocarbonylmethyl-, or diC₁₋₄alkylaminocarbonylmethyl-.

In certain embodiments of any of the above formulae, ZR₁ is 3,3 diphenylpropyl optionally substituted at the 3 carbon of the propyl with —COOV₁, tetrazolylC₀₋₄alkyl-, cyano-, aminocarbonyl-, C₁₋₄alkylaminocarbonyl-, or diC₁₋₄alkylaminocarbonyl-.

In the most preferred embodiment of the invention, ZR₁ is cyclooctyl.

In preferred embodiments, the compound formed is 1-(1-Cyclooctyl-piperidin-4-yl)-1,3-dihydro-benzoimidazol-2-ylidene-cyanamide or 2-[2-Cyanoimino-3-(1-cyclooctyl-piperidin-4-yl)-2,3-dihydro-benzoimidazol-1-yl]-acetamide

As used herein, the term “alkyl” means a linear or branched saturated aliphatic hydrocarbon group having a single radical and 1-10 carbon atoms. Examples of alkyl groups include methyl, propyl, isopropyl, butyl, n-butyl, isobutyl, sec-butyl, tert-butyl, and pentyl. A branched alkyl means that one or more alkyl groups such as methyl, ethyl or propyl, replace one or both hydrogens in a —CH₂— group of a linear alkyl chain. The term “lower alkyl” means an alkyl of 1-3 carbon atoms.

The term “alkoxy” means an “alkyl” as defined above connected to an oxygen radical.

The term “cycloalkyl” means a non-aromatic mono- or multicyclic hydrocarbon ring system having a single radical and 3-12 carbon atoms. Exemplary monocyclic cycloalkyl rings include cyclopropyl, cyclopentyl, and cyclohexyl. Exemplary multicyclic cycloalkyl rings include adamantyl and norbornyl.

The term “alkenyl” means a linear or branched aliphatic hydrocarbon group containing at least one carbon-carbon double bond having a single radical and 2-10 carbon atoms. A “branched” alkenyl means that one or more alkyl groups such as methyl, ethyl or propyl replace one or both hydrogens in a —CH₂— or —CH═ linear alkenyl chain. Exemplary alkenyl groups include ethenyl, 1- and 2-propenyl, 1-, 2- and 3-butenyl, 3-methylbut-2-enyl, 2-propenyl, heptenyl, octenyl and decenyl.

The term “cycloalkenyl” means a non-aromatic monocyclic or multicyclic hydrocarbon ring system containing at least one carbon-carbon double bond having a single radical and 3 to 12 carbon atoms. Exemplary monocyclic cycloalkenyl rings include cyclopropenyl, cyclopentenyl, cyclohexenyl and cycloheptenyl. An exemplary multicyclic cycloalkenyl ring is norbornenyl.

The term “aryl” means a carbocyclic aromatic ring system containing one, two or three rings which may be attached together in a pendent manner or fused, and containing a single radical. Exemplary aryl groups include phenyl, naphthyl and acenaphthyl.

The term “heterocyclic” means cyclic compounds having one or more heteroatoms (atoms other than carbon) in the ring, and having a single radical. The ring may be saturated, partially saturated or unsaturated, and the heteroatoms may be selected from the group consisting of nitrogen, sulfur and oxygen. Examples of saturated heterocyclic radicals include saturated 3 to 6-membered hetero-monocyclic groups containing 1 to 4 nitrogen atoms, such as pyrrolidinyl, imidazolidinyl, piperidino, piperazinyl; saturated 3- to 6-membered hetero-monocyclic groups containing 1 to 2 oxygen atoms and 1 to 3 nitrogen atoms, such as morpholinyl; saturated 3- to 6-membered hetero-monocyclic groups containing 1 to 2 sulfur atoms and 1 to 3 nitrogen atoms, such as thiazolidinyl. Examples of partially saturated heterocyclic radicals include dihydrothiophene, dihydropyran, and dihydrofuran. Other heterocyclic groups can be 7 to 10 carbon rings substituted with heteroatoms such as oxocanyl and thiocanyl. When the heteroatom is sulfur, the sulfur can be a sulfur dioxide such as thiocanyldioxide.

The term “heteroaryl” means unsaturated heterocyclic radicals, wherein “heterocyclic” is as previously described. Exemplary heteroaryl groups include unsaturated 3 to 6 membered hetero-monocyclic groups containing 1 to 4 nitrogen atoms, such as pyrrolyl, pyridyl, pyrimidyl, and pyrazinyl; unsaturated condensed heterocyclic groups containing 1 to 5 nitrogen atoms, such as indolyl, quinolyl and isoquinolyl; unsaturated 3 to 6-membered hetero-monocyclic groups containing an oxygen atom, such as furyl; unsaturated 3 to 6 membered hetero-monocyclic groups containing a sulfur atom, such as thienyl; unsaturated 3 to 6 membered hetero-monocyclic groups containing 1 to 2 oxygen atoms and 1 to 3 nitrogen atoms, such as oxazolyl; unsaturated condensed heterocyclic groups containing 1 to 2 oxygen atoms and 1 to 3 nitrogen atoms, such as benzoxazolyl; unsaturated 3 to 6 membered hetero-monocyclic groups containing 1 to 2 sulfur atoms and 1 to 3 nitrogen atoms, such as thiazolyl; and unsaturated condensed heterocyclic group containing 1 to 2 sulfur atoms and 1 to 3 nitrogen atoms, such as benzothiazolyl. The term “heteroaryl” also includes unsaturated heterocyclic radicals, wherein “heterocyclic” is as previously described, in which the heterocyclic group is fused with an aryl group, in which aryl is as previously described. Exemplary fused radicals include benzofuran, benzdioxole and benzothiophene.

As used herein, the term “heterocyclicC₁₋₄alkyl”, “heteroaromaticC₁₋₄alkyl” and the like refer to the ring structure bonded to a C₁₋₄ alkyl radical.

All of the cyclic ring structures disclosed herein can be attached at any point where such connection is possible, as recognized by one skilled in the art.

As used herein, the term “patient” includes a human or an animal such as a companion animal or livestock.

As used herein, the term “halogen” includes fluoride, bromide, chloride, iodide or alabamide.

The processes of the invention can further comprise preparing a pharmaceutically acceptable acid addition salt of the prepared compounds.

The compositions disclosed herein can also be in the form of a pharmaceutically acceptable salt, e.g., an acid addition salt.

The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, fumarate, tartrate and the like; sulfonates such as methanesulfonate, benzenesulfonate, p-toluenesulfonate, and the like; amino acid salts such as arginate, asparginate, glutamate and the like. The most preferred salt is the hydrochloride salt.

### DETAILED DESCRIPTION

In certain embodiments, the invention is directed to a process for synthesizing a compound of formula (VI):

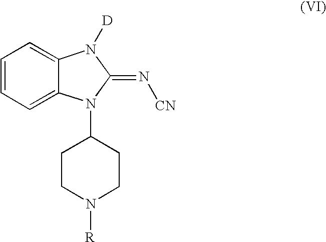

wherein D is selected from the group consisting of C₁₋₁₀ alkyl, C₃₋₁₂ cycloalkyl, C₃₋₁₂ cycloalkylC₁₋₄alkyl-, C₁₋₁₀ alkoxy, C₃₋₁₂ cycloalkoxy-, C₁₋₁₀ alkyl substituted with 1-3 halogen, C₃₋₁₂ cycloalkyl substituted with 1-3 halogen, C₃₋₁₂ cycloalkylC₁₋₄alkyl-substituted with 1-3 halogen, C₁₋₁₀ alkoxy substituted with 1-3 halogen, C₃₋₁₂ cycloalkoxy-substituted with 1-3 halogen, —COOV₁, —C₁₋₄COOV₁, —CH₂OH, —SO₂N(V₁)₂, hydroxyC₁₋₁₀alkyl-, hydroxyC₃₋₁₀cycloakyl-, cyanoC₁₋₁₀alkyl-, cyanoC₃₋₁₀cycloalkyl-, —CON(V₁)₂, NH₂SO₂C₁₋₄alkyl-, NH₂SOC₁₋₄allyl-, sulfonylaminoC₁₋₁₀alkyl-, diaminoalkyl-, -sulfonylC₁₋₄alkyl, a 6-membered heterocyclic ring, a 6-membered heteroaromatic ring, a 6-membered heterocyclicC₁₋₄alkyl-, a 6-membered heteroaromaticC₁₋₄alkyl-, a 6-membered aromatic ring, a 6-membered aromaticC₁₋₄ alkyl-, a 5-membered heterocyclic ring optionally substituted with an oxo or thio, a 5-membered heteroaromatic ring, a 5-membered heterocyclicC₁₋₄alkyl-optionally substituted with an oxo or thio, a 5-membered heteroaromaticC₁₋₄alkyl-, —C₁₋₅(═O)W₁, —C₁₋₅(═NH)W₁, —C₁₋₅NHC(═O)W₁, —C₁₋₅NHS(═O)₂W₁—C₁₋₅NHS(═O)W₁,

R is -Z-R1, wherein

Z is selected from the group consisting of a bond, straight or branched C₁₋₆ alkylene, —NH—, —CH₂O—, —CH₂NH—, —CH₂N(CH₃)—, —NHCH₂—, —CH₂CONH—, —NHCH₂CO—, —CH₂CO—, —COCH₂—, —CH₂COCH₂—, —CH(CH₃)—, —CH═, —O— and —HC═CH—, wherein the carbon and/or nitrogen atoms are unsubstituted or substituted with one or more lower alkyl, hydroxy, halo or alkoxy group;

R₁ is selected from the group consisting of hydrogen, C₁₋₁₀ alkyl, C₃₋₁₂cycloalkyl, C₂₋₁₀alkenyl, amino, C₁₋₁₀alkylamino-, C₃₋₁₂cycloalkylamino-, —COOV₁, —C₁₋₄COOV₁, cyano, cyanoC₁₋₁₀alkyl-, cyanoC₃₋₁₀cycloalkyl-, NH₂SO₂—, NH₂SO₂C₁₋₄alkyl-, NH₂SOC₁₋₄alkyl-, aminocarbonyl-, C₁₋₄alkylaminocarbonyl-, diC₁₋₄alkylaminocarbonyl-, benzyl, C₃₋₁₂ cycloalkenyl-, a monocyclic, bicyclic or tricyclic aryl or heteroaryl ring, a hetero-monocyclic ring, a hetero-bicyclic ring system, and a spiro ring system of the formula (XI):

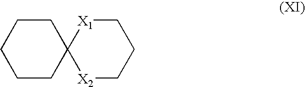

wherein X₁ and X₂ are independently selected from the group consisting of NH, O, S and CH₂; and wherein said alkyl, cycloalkyl, alkenyl, C₁₋₁₀alkylamino-, C₃₋₁₂cycloalkylamino-, or benzyl of R₁ is optionally substituted with 1-3 substituents selected from the group consisting of halogen, hydroxy, C₁₋₁₀ alkyl, C₁₋₁₀ alkoxy, nitro, trifluoromethyl-, cyano, —COOV₁, —C₁₋₄COOV₁, cyanoC₁₋₁₀alkyl-, —C₁₋₅(═O)W₁, —C₁₋₅NHS(═O—)₂W₁, —C₁₋₅NHS(═O)W₁, a 5-membered heteroaromaticC₀₋₄alkyl-, phenyl, benzyl, benzyloxy, said phenyl, benzyl, and benzyloxy optionally being substituted with 1-3 substituents selected from the group consisting of halogen, C₁₋₁₀ alkyl-, C₁₋₁₀ alkoxy-, and cyano; and wherein said C₃₋₁₂ cycloalkyl, C₃₋₁₂ cycloalkenyl, monocyclic, bicyclic or tricyclic aryl, heteroaryl ring, hetero-monocyclic ring, hetero-bicyclic ring system, or spiro ring system of the formula (XI) is optionally substituted with 1-3 substituents selected from the group consisting of halogen, C₁₋₁₀ alkyl, C₁₋₁₀ alkoxy, nitro, trifluoromethyl-, phenyl, benzyl, phenyloxy and benzyloxy, wherein said phenyl, benzyl, phenyloxy or benzyloxy is optionally substituted with 1-3 substituents selected from the group consisting of halogen, C₁₋₁₀ alkyl, C₁₋₁₀ alkoxy, and cyano;

wherein each V₁ is independently selected from H, C₁₋₆ alkyl, C₃₋₆ cycloalkyl, benzyl and phenyl; and

wherein W₁ is hydrogen, C₁₋₁₀ alkyl, C₃₋₁₂ cycloalkyl, C₁₋₁₀ alkoxy, C₃₋₁₂ cycloalkoxy, —CH₂OH, amino, C₁₋₄alkylamino-, diC₁₋₄alkylamino-, or a 5-membered heteroaromatic ring optionally substituted with 1-3 lower alkyl.

comprising reacting a compound of formula (V):

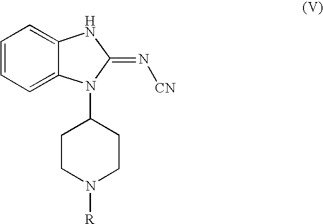

with a D-halogen to form a compound of formula (VI), wherein R is as disclosed herein.

In certain embodiments, D is —CH₂CONH₂.

In certain embodiments, the halogen is bromide.

In certain embodiments, the reaction to prepare a compound of formula (VI) can be performed in a suitable solvent, e.g., a solvent selected from tetrahydrofuran, dimethylformamide, or a mixture thereof.

In certain embodiments, the reaction to prepare a compound of formula (VI) can be initiated at ambient temperature and raised to a temperature, e.g., of about 50° C. or less. Preferably, the reaction is performed at a temperature from about 20° to about 35° C. or about 25° to about 30° C.

In certain embodiments, the invention is directed to a process for synthesizing a compound of formula (V):

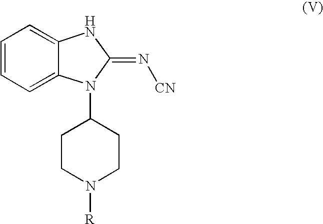

comprising reacting a compound of formula (IV)

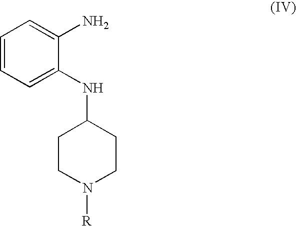

with (A)(A₁)-cyanocarbonimidate to form a compound of formula V;

wherein A and A₁ are independently selected from methyl, ethyl propyl, phenyl and benzyl; and wherein R is as disclosed herein.

In certain embodiments, A and A₁ are both phenyl.

In certain embodiments, the reaction to prepare a compound of formula (V) can be performed in a suitable solvent, e.g., a solvent selected from acetonitrile, dimethylformamide, or a mixture thereof.

In certain embodiments, the reaction to prepare a compound of formula (V) can be performed at a temperature of about 50° C. to about 120° C. or about 75° C. to about 125° C. or about 100° C.

In certain embodiments of preparing a compound of formula (V), a portion of the reaction is performed under ambient temperature.

In certain embodiments of preparing a compound of formula (V), an intermediate cyanoimidate (as depicted below) is isolated.

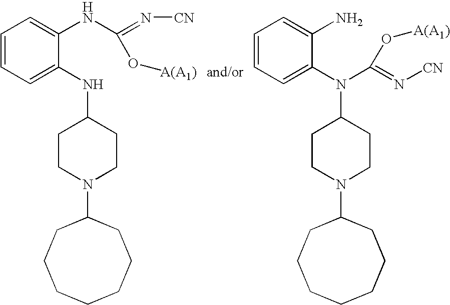

In other embodiments, the reaction is conducted as a “one pot reaction” in a solvent such as acetonitrile, dimethylformamide or a mixture thereof.

In certain embodiments, the invention is directed to a process for synthesizing a compound of formula (IV) by subjecting a compound of formula (III):

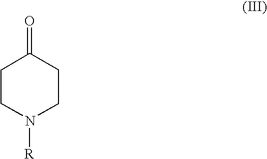

wherein R is as disclosed herein,

to reductive amination with 1,2-phenylenediamine, an acid and a reducing agent to form a compound of formula (IV).

In certain embodiments of preparing a compound of formula (IV), the reductive amination is performed in a suitable solvent, e.g., dichloroethane, tetrahydrofuran, any suitable acidic solvent known to one skilled in the art, or a mixture thereof.

In certain embodiments of preparing a compound of formula (IV), the acid is acetic acid, proprionic acid, paratoluenesulfonic acid, any suitable acid known to one skilled in the art to catalyze the reaction, or a mixture thereof.

In certain embodiments of preparing a compound of formula (IV), the reducing agent is selected from the group consisting of sodium triacetoxyborohydride, sodium acetoxyborohydride, sodium borohydride, lithium borohydride, lithium aluminum hydride and a combination thereof. Preferably, the reducing agent is lithium aluminum hydride.

In certain embodiments of preparing a compound of formula (IV), the reductive amination is performed at ambient temperature.

In certain embodiments, the compounds of formula (IV) can be prepared by an alternative process by subjecting a compound of formula (III):

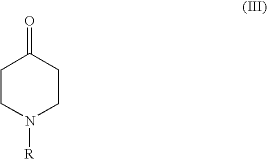

wherein R is as disclosed herein,

to amination with 1,2-phenylenediamine and an acid to form a compound of, formula (IIIA):

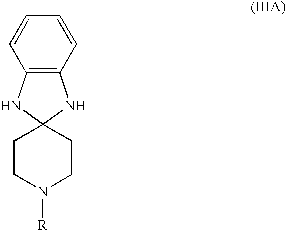

wherein R is as disclosed herein,

and reducing the compound of (IIIA) with a reducing agent to form a compound of formula (IV).

In certain embodiments of the alternative process of preparing a compound of formula (IV), the amination is performed in a suitable solvent, e.g., dichloroethane, tetrahydrofuran, any suitable acidic solvent known to one skilled in the art, or a mixture thereof.

In certain embodiments of the alternative process of preparing a compound of formula (IV), the acid is acetic acid, proprionic acid, paratoluenesulfonic acid, any suitable acid known to one skilled in the art to catalyze the reaction, or a mixture thereof.

In certain embodiments of the alternative process of preparing a compound of formula (IV), the compound of formula IIIA is recovered.

In certain embodiments of the alternative process of preparing a compound of formula (IV), the compound of formula IIIA is recovered as a gum.

In certain embodiments of the alternative process of preparing a compound of formula (IV), the recovered compound is dissolved in a solvent and reduced with the reducing agent.

In certain embodiments of the alternative process of preparing a compound of formula (IV), the reducing agent is selected from the group consisting of sodium triacetoxyborohydride, sodium acetoxyborohydride, sodium borohydride, lithium borohydride, lithium aluminum hydride and a combination thereof. Preferably, the reducing agent is lithium aluminum hydride.

In certain embodiments of the alternative process of preparing a compound of formula (IV), the reduction is initiated at a temperature below about 10° C. and raised to a temperature of about 30° C. to about 70° C. or about 50° C. to about 65° C. in an ethereal solvent, e.g., tetrahydrofuran.

In certain embodiments, the invention is directed to a process for synthesizing a compound of formula (III) by reacting a compound of formula (II):

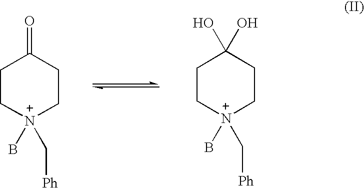

with R-amine to form a compound of formula III;

wherein B is selected from the group consisting of methyl, ethyl and propyl and R is as disclosed herein. In certain embodiments, depending of factors such as the solvent utilized, the ratio of the oxo compound of formula II to the dihydroxy compound of formula II is from 100:0 to 0:100; from 90:10 to 10:90; from 75:25 to 25:75 or about 50:50.

In certain embodiments, the compounds of formula (III) can be prepared by the alternative process of reacting a compound of formula (IIA):

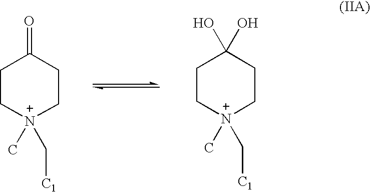

with R-amine to form a compound of formula III, wherein C and C₁ are independently selected from the group consisting of methyl, ethyl and propyl and wherein R is as disclosed herein. In certain embodiments, depending of factors such as the solvent utilized, the ratio of the oxo compound of formula IIA to the dihydroxy compound of formula IIA is from 100:0 to 0:100, from 90:10 to 10:90; from 75:25 to 25:75 or about 50:50.

In certain embodiments, the invention is directed to another alternative process for synthesizing a compound of formula (III) by reacting a compound of formula (IIB):

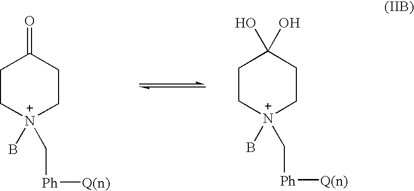

with R-amine to form a compound of formula III;

wherein B is selected from the group consisting of methyl, ethyl and propyl; R is as disclosed herein; Q is a member selected from the group consisting of COOH, C₁₋₃alkyl, halogen, haloC₁₋₃alkyl, hydroxyl and nitro; and n an integer from 1-3. In certain embodiments, depending of factors such as the solvent utilized, the ratio of the oxo compound of formula IIB to the dihydroxy compound of formula IIB is from 100:0 to 0:100; from 90:10 to 10:90; from 75:25 to 25:75 or about 50:50.

The formation of the compound of formula (III) utilizing a compound of formula (IIB) is preferred than utilizing a compound of formula (II) due to increased yield and the facilitation of recovery of the intended product.

For example, reaction of a compound of formula (II) with an R-amine results in the formation of a compound of formula (III) and the byproduct of formula (VII):

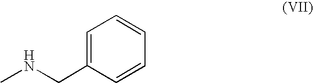

The purification of the compound of formula (III) from formula (VII) is complicated by the fact that the compound of formula (VII) co-distills with formula (III). Another attempt at this purification requires chromatography.

The modification of the phenyl group of formula (II) to arrive at the compound of formula (IIB) results in a corresponding modification of the byproduct compound of formula (VIIA).

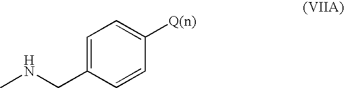

wherein Q and n are as defined above.

The purification of the compound of formula (III) from formula (VIIA) is facilitated as compared to purification from formula (VII). The compound of formula (VIIA) is soluble in basic aqueous media. Therefore, by running the reaction in a basic pH, or by adjusting the pH of the media to be basic during or after the reaction (e.g., to a pH of >8), the compound of formula (III) which can be recovered, e.g., by biphasic partition. The partition can be performed e.g., with a organic/aqueous solvent such as a hexane/water solvent.

In other purification techniques, the Q substituent has an acidic tail and an ion resin can be used to purify (e.g., by filtration) a mixture of a compound of formula (III) and (VIIA).

In other purification techniques, the compound of formula (VIIA) can be converted during the reaction or during pH adjustment to be hydrophobic, whereby it will dissolve in organic solvent. This can be performed, e.g., by modifying Q to be e.g., a COOH group or an ester group which is ortho to the amine. In such embodiments, a compound of formula (III) can be subject to biphasic partition.

In certain embodiments of preparing a compound of formula (III), the reaction is performed in a suitable solvent, e.g., an alcohol, water or a mixture thereof. In certain embodiments, the solvent is ethanol and water. The reaction can be performed at a temperature, e.g., from about 50° C. to about 120° C. In certain embodiments, the reaction can be performed under reflux conditions.

In certain embodiments, the compounds of formula (II) can be prepared by reacting a compound of formula (I):

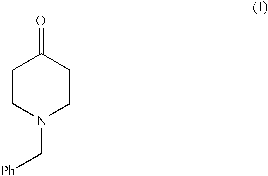

with an appropriate C₁₋₃alkyl-halogen to form a compound of formula II.

In certain embodiments of preparing a compound of formula (II) with a compound of formula (I), the C₁₋₃alkyl-halogen is iodomethane.

In certain embodiments of preparing a compound of formula (II) with a compound of formula (I), the reaction is performed in a suitable solvent such as acetone, ethyl acetate, ethereal solvents, toluene, hexane, cyclohexane, and mixtures thereof. The reaction can be performed under reflux conditions.

In certain embodiments, the compounds of formula (II) can be prepared by reacting a compound of formula (IA):

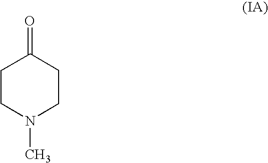

with a benzyl-halogen to form a compound of formula II.

In certain embodiments of preparing a compound of formula (II) with a compound of formula (IA), the halogen is bromide.

In certain embodiments of preparing a compound of formula (II) with a compound of formula (IA), the reaction is performed in a suitable solvent such as acetone ethyl acetate, ethereal solvents, toluene, hexane, cyclohexane, and mixtures thereof. The reaction can be performed under reflux conditions.

In certain embodiments, the compounds of formula (IIA) can be prepared by an alternative process by reacting a compound of formula (IA):

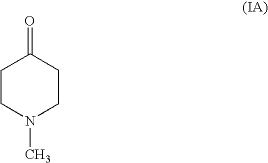

with (C)(C₁)sulphate to form a compound of formula IIA.

In certain embodiments of preparing a compound of formula (IIA) with the alternative process utilizing a compound of formula (IA), C and C₁ are both methyl.

In certain embodiments of preparing a compound of formula (IIA) with the alternative process utilizing a compound of formula (IA), the reaction is performed in a suitable solvent, e.g., acetone, ethyl acetate, ethereal solvents, toluene, hexane, cyclohexane, and mixtures thereof. In certain embodiments, the compound of formula IA and the solvent are cooled to a temperature below 10° C. prior to the addition of the (C)(C₁)sulphate.

In certain embodiments, the invention is further directed to converting a compound of formula (V) or (VI) to a pharmaceutically acceptable salt, e.g., an acid addition salt.

In certain embodiments, the process of the present invention comprises preparing a compound of formula (VI) from a compound of formula (I); from a compound of formula (IA); from a compound of formula (II); from a compound of formula (IIA); from a compound of formula (III); from a compound of formula (IIIA); from a compound of formula (IV); or from a compound of formula (V); utilizing the step(s) disclosed above.

In certain embodiments, the process of the present invention comprises preparing a compound of formula (V) from a compound of formula (I); from a compound of formula (IA); from a compound of formula (II); from a compound of formula (IIA); from a compound of formula (III); from a compound of formula (IIIA); or from a compound of formula (IV); utilizing the step(s) disclosed above.

In certain embodiments, the process of the present invention comprises preparing a compound of formula (IV) from a compound of formula (I); from a compound of formula (IA); from a compound of formula (II); from a compound of formula (IIA); from a compound of formula (III); or from a compound of formula (IIIA); utilizing the step(s) disclosed above.

In certain embodiments, the process of the present invention comprises preparing a compound of formula (IIIA) from a compound of formula (I); from a compound of formula (IA); from a compound of formula (II); from a compound of formula (IIA); or from a compound of formula (III) utilizing the step(s) disclosed above.

In certain embodiments, the process of the present invention comprises preparing a compound of formula (III) from a compound of formula (I); from a compound of formula (IA); from a compound of formula (II); or from a compound of formula (IIA) utilizing the step(s) disclosed above.

When the present invention is directed to compounds, e.g., 1-(1-cyclooctyl-piperidin-4-yl)-1,3-dihydro-benzoimidazol-2-ylidene-cyanamide, the compounds of the present invention can be administered to anyone requiring agonization of the ORL1 receptors. Administration may be orally, topically, by suppository, inhalation, or parenterally.

The present invention also encompasses all pharmaceutically acceptable salts of the compounds. One skilled in the art will recognize that acid addition salts of the presently claimed compounds may be prepared by reaction of the compounds with the appropriate acid via a variety of known methods.

Various oral dosage forms can be used, including such solid forms as tablets, gelcaps, capsules, caplets, granules, lozenges and bulk powders and liquid forms such as emulsions, solution and suspensions. The compounds of the present invention can be administered alone or can be combined with various pharmaceutically acceptable carriers and excipients known to those skilled in the art, including but not limited to diluents, suspending agents, solubilizers, binders, disintegrants, preservatives, coloring agents, lubricants and the like.

When the compounds of the present invention are incorporated into oral tablets, such tablets can be compressed, tablet triturates, enteric-coated, sugar-coated, film-coated, multiply compressed or multiply layered. Liquid oral dosage forms include aqueous and nonaqueous solutions, emulsions, suspensions, and solutions and/or suspensions reconstituted from non-effervescent granules, containing suitable solvents, preservatives, emulsifying agents, suspending agents, diluents, sweeteners, coloring agents, and flavoring agents. When the compounds of the present invention are to be injected parenterally, they may be, e.g., in the form of an isotonic sterile solution. Alternatively, when the compounds of the present invention are to be inhaled, they may be formulated into a dry aerosol or may be formulated into an aqueous or partially aqueous solution.

In addition, when the compounds of the present invention are incorporated into oral dosage forms, it is contemplated that such dosage forms may provide an immediate release of the compound in the gastrointestinal tract, or alternatively may provide a controlled and/or sustained release through the gastrointestinal tract. A wide variety of controlled and/or sustained release formulations are well known to those skilled in the art, and are contemplated for use in connection with the formulations of the present invention. The controlled and/or sustained release may be provided by, e.g., a coating on the oral dosage form or by incorporating the compound(s) of the invention into a controlled and/or sustained release matrix.

Specific examples of pharmaceutically acceptable carriers and excipients that may be used to formulate oral dosage forms, are described in the Handbook of Pharmaceutical Excipients, American Pharmaceutical Association (1986). Techniques and compositions for making solid oral dosage forms are described in Pharmaceutical Dosage Forms: Tablets (Lieberman, Lachman and Schwartz, editors) 2nd edition, published by Marcel Dekker, Inc. Techniques and compositions for making tablets (compressed and molded), capsules (hard and soft gelatin) and pills are also described in Remington's Pharmaceutical Sciences (Arthur Osol, editor), 1553B1593 (1980). Techniques and composition for making liquid oral dosage forms are described in Pharmaceutical Dosage Forms: Disperse Systems, (Lieberman, Rieger and Banker, editors) published by Marcel Dekker, Inc.

When the compounds of the present invention are incorporated for parenteral administration by injection (e.g., continuous infusion or bolus injection), the formulation for parenteral administration may be in the form of suspensions, solutions, emulsions in oily or aqueous vehicles, and such formulations may further comprise pharmaceutically necessary additives such as stabilizing agents, suspending agents, dispersing agents, and the like. The compounds of the invention may also be in the form of a powder for reconstitution as an injectable formulation.

In certain embodiments, the compounds of the present invention can be used in combination with at least one other therapeutic agent. Therapeutic agents include, but are not limited to, μ-opioid agonists; non-opioid analgesics; non-steroid antiinflammatory agents; Cox-II inhibitors; antiemetics; β-adrenergic blockers; anticonvulsants; antidepressants; Ca2+-channel blockers; anticancer agent and mixtures thereof.

In certain embodiments, the compounds of the present invention can be formulated in a pharmaceutical dosage form in combination with a μ-opioid agonist. μ-opioid agonists, which may be included in the formulations of the present invention include but are not limited to include alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof and mixtures thereof.

In certain preferred embodiments, the μ-opioid agonist is selected from codeine, hydromorphone, hydrocodone, oxycodone, dihydrocodeine, dihydromorphine, morphine, tramadol, oxymorphone, pharmaceutically acceptable salts thereof, and mixtures thereof.

In another embodiment of the invention, the medicament comprises a mixture of a Cox-II inhibitor and an inhibitor of 5-lipoxygenase for the treatment of pain and/or inflammation. Suitable Cox-II inhibitors and 5-lipoxygenase inhibitors, as well as combinations thereof are described in U.S. Pat. No. 6,136,839, which is hereby incorporated by reference in its entirety. Cox-II inhibitors include, but are not limited to rofecoxib (Vioxx), celecoxib (Celebrex), DUP-697, flosulide, meloxicam, 6-MNA, L-745337, nabumetone, nimesulide, NS-398, SC-5766, T-614, L-768277, GR-253035, JTE-522, RS-57067-000, SC-58125, SC-078, PD-138387, NS-398, flosulide, D-1367, SC-5766, PD-164387, etoricoxib, valdecoxib and parecoxib or pharmaceutically acceptable salts, enantiomers or tautomers thereof.

The compounds of the present invention can also be combined in dosage forms with non-opioid analgesics, e.g., non-steroidal anti-inflammatory agents, including aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid tolfenamic acid, diflurisal, flufenisal, piroxicam, sudoxicam or isoxicam, pharmaceutically acceptable salts thereof and mixtures thereof. Other suitable non-opioid analgesics which may be included in the dosage forms of the present invention include the following, non-limiting, chemical classes of analgesic, antipyretic, nonsteroidal antiinflammatory drugs: salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone. For a more detailed description of the NSAIDs that may be included within the medicaments employed in the present invention, see Paul A. Insel Analgesic-Antipyretic and Antiinflammatory Agents and Drugs Employed in the treatment of Gout in Goodman & Gilman's The Pharmacological Basis of Therapeutics, 617-57 (Perry B. Molinhoff and Raymond W. Ruddon, Eds., Ninth Edition, 1996), and Glen R. Hanson Analgesic, Antipyretic and Anti-Inflammatory Drugs in Remington: The Science and Practice of Pharmacy Vol II, 1196-1221 (A. R. Gennaro, Ed. 19th Ed. 1995).

In certain embodiments, the compounds of the present invention can be formulated in a pharmaceutical dosage form in combination with antimigraine agents. Antimigraine agents include, but are not limited to, alpiropride, dihydroergotamine, dolasetron, ergocornine, ergocorninine, ergocryptine, ergot, ergotamine, flumedroxone acetate, fonazine, lisuride, lomerizine, methysergide oxetorone, pizotyline, and mixtures thereof.

The other therapeutic agent can also be an adjuvant to reduce any potential side effects such as, for example, an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine, tropisetron, and mixtures thereof.

In certain embodiments, the compounds of the present invention can be formulated in a pharmaceutical dosage form in combination with β-adrenergic blockers. Suitable β-adrenergic blockers include, but are not limited to, acebutolol, alprenolol, amosulabol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol, indenolol, labetalol, levobunolol, mepindolol, metipranolol, metoprolol, moprolol, nadolol, nadoxolol, nebivalol, nifenalol, nipradilol, oxprenolol, penbutolol, pindolol, practolol, pronethalol, propranolol, sotalol, sulfinalol, talinolol, tertatolol, tilisolol, timolol, toliprolol, and xibenolol.

In certain embodiments, the compounds of the present invention can be formulated in a pharmaceutical dosage form in combination with anticonvulsants. Suitable anticonvulsants include, but are not limited to, acetylpheneturide, albutoin, aloxidone, aminoglutethimide, 4-amino-3-hydroxybutyric acid, atrolactamide, beclamide, buramate, calcium bromide, carbamazepine, cinromide, clomethiazole, clonazepam, decimemide, diethadione, dimethadione, doxenitroin, eterobarb, ethadione, ethosuximide, ethotoin, felbamate, fluoresone, gabapentin, 5-hydroxytryptophan, lamotrigine, magnesium bromide, magnesium sulfate, mephenyloin, mephobarbital, metharbital, methetoin, methsuximide, 5-methyl-5-(3-phenanthryl)-hydantoin, 3-methyl-5-phenylhydantoin, narcobarbital, nimetazepam, nitrazepam, oxcarbazepine, paramethadione, phenacemide, phenetharbital, pheneturide, phenobarbital, phensuximide, phenylmethylbarbituric acid, phenyloin, phethenylate sodium, potassium bromide, pregabaline, primidone, progabide, sodium bromide, solanum, strontium bromide, suclofenide, sulthiame, tetrantoin, tiagabine, topiramate, trimethadione, valproic acid, valpromide, vigabatrin, and zonisamide.

In certain embodiments, the compounds of the present invention can be formulated in a pharmaceutical dosage form in combination with antidepressants. Suitable antidepressants include, but are not limited to, binedaline, caroxazone, citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, pheneizine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

In certain embodiments, the compounds of the present invention can be formulated in a pharmaceutical dosage form in combination with Ca2+-channel blockers. Suitable Ca2+ channel blockers include, but are not limited to, bepridil, clentiazem, diltiazem, fendiline, gallopamil, mibefradil, prenylamine, semotiadil, terodiline, verapamil, amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, fantofarone, and perhexiline.

In certain embodiments, the compounds of the present invention can be formulated in a pharmaceutical dosage form in combination with anticancer agents. Suitable anticancer agents include, but are not limited to, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; talliimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

The compounds of the present invention and the other therapeutic agent can act additively or, more preferably, synergistically. In a preferred embodiment, a composition-comprising a compound of the present invention is administered concurrently with the administration of another therapeutic agent, which can be part of the same composition or in a different composition from that comprising the compounds of the present invention.

In another embodiment, a composition comprising the compounds of the present invention is administered prior to or subsequent to administration of the other therapeutic agent.

The compounds of the present invention when administered, e.g., via the oral, parenteral or topical routes to mammals, can be in a dosage in the range of about 0.01 mg/kg to about 3000 mg/kg body weight of the patient per day, preferably about 0.01 mg/kg to about 1000 mg/kg body weight per day administered singly or as a divided dose. However, variations will necessarily occur depending upon the weight and physical condition (e.g., hepatic and renal function) of the subject being treated, the affliction to be treated, the severity of the symptoms, the route of administration, the frequency of the dosage interval, the presence of any deleterious side-effects, and the particular compound utilized, among other things.

### EXAMPLE 1

### Preparation of 1-(1-Cyclooctyl-piperidin-4-yl)-1,3-dihydro-benzoimidazol-2-ylidene-cyanamide and 2-[2-Cyanoimino-3-(1-cyclooctyl-piperidin-4-yl)-2,3-dihydro-benzoimidazol-1-yl]-acetamide

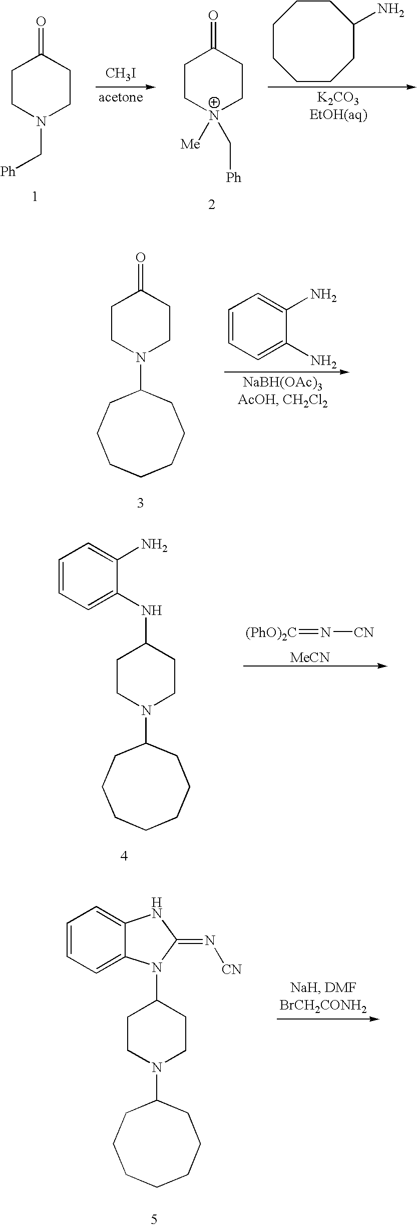

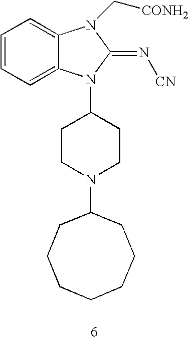

### 1-Benzyl-1-methyl-4-oxo-piperidinium iodide (2)

1-Benzyl-4-piperidone (100 g, 528 mmol) was dissolved in acetone (600 mL). Iodomethane (32.9 mL, 528 mmol) was added and the mixture heated under reflux for 2 h. The cooled mixture was filtered, and the solid washed with ether and dried in vacuo to give the desired product (131 g, 75%) as a white solid.

¹H-NMR (400 MHz, d⁶-DMSO) δ 7.60 (m, 5H), 4.75 (s, 2H), 3.75 (m, 2H), 3.65 (m, 2H), 3.10 (s, 3H) 2.85 (m, 2H), 2.70 (m, 2H).

### 1-Cyclooctyl-piperidin-4-one (3)

Cyclooctylamine (23.8 mL, 177.8 mmol) was dissolved in ethanol (250 mL), potassium carbonate (3.68 g, 26.4 mmol) was added and the mixture was brought to reflux. 1-Benzyl-1-methyl-4-oxo-piperidinium iodide (87.6 g, 264.4 mmol) was dissolved in a boiling solution of water:ethanol (120 mL:280 mL), and the solution added slowly over 30 min. The resulting solution was stirred at reflux for an additional 1 h. The cooled mixture was evaporated to dryness in vacuo, and the residue partitioned between 0.1M sodium hydroxide solution (1 L) and ether (1 L). The organic phase was separated, dried (MgSO₄) and evaporated to dryness in vacuo to leave a yellow oil. Flash chromatography eluting with ether gave the desired product (24.5 g, 66%) as a pale yellow oil. TLC (SiO₂, ether) Rf=0.60 (Dragendorff's Reagent)

¹H-NMR (400 MHz, CDCl₃) δ 2.80 (t, 2H, J=6 Hz), 2.43 (t, 2H, J=6 Hz), 1.7 (m, 2H), 1.65-1.40 (m, 13H).

### N-(1-Cyclooctyl-piperidin-4-yl)-benzene-1,2-diamine (4)

1,2-Phenylenediamine (12.4 g, 114.65 mmol) and 1-cyclooctyl-piperidin-4-one (8.0 g, 38.2 mmol) were dissolved in 1,2-dichloroethane (100 mL). Acetic acid (3 mL) was added followed by sodium triacetoxyborohydride (12.14 g, 57.3 mmol) and the mixture stirred overnight at room temperature. The solvent was removed in vacuo, and the residue was partitioned between ether (500 mL) and 2M sodium carbonate solution (500 mL). The organic phase was separated, dried (MgSO₄) and the solvent evaporated to dryness in vacuo to leave an orange gum. Flash chromatography, eluting with ethyl acetate (2 L) followed by ethyl acetate:methanol:ammonia (100:10:1) gave the desired product 4 (9.3 g, 81%) as a pale orange solid.

¹H-NMR (400 MHz, CDCl₃) δ 6.8 (t, 1H, J=12 Hz), 6.75 (d, 1H, J=12 Hz), 6.65 (m, 2H), 3.3 (bs, 1H), 3.2 (bs, 2H), 2.8 (bd, 2H), 2.60 (bt, 1H), 2.35 (t, J=10 Hz, 2H), 2.1 (d, 2H, J=10 Hz), 1.80-1.40 (m, 15H).

### 1-(1-Cyclooctyl-piperidin-4-yl)-1,3-dihydro-benzoimidazol-2-ylidene-cyanamide (5)

The triamine 4 (9.5 g, 31.5 mmol) was dissolved in acetonitrile (100 mL). Diphenyl cyanocarbonimidate (8.25 g, 34.65 mmol) was added and the mixture heated under reflux for 2 h. The mixture was filtered to give the intermediate cyanoimidate (10.8 g). This was suspended in dry N,N-dimethylformamide (150 mL) and heated under reflux for 6 h. The cooled mixture was evaporated to dryness to leave a pale yellow solid. This was triturated with ethyl acetate (100 mL) to give the desired product (6.5 g, 59%) as a white solid.

Alternatively, the reaction can be run in one pot in acetonitrile for 3-4 days at reflux, eliminating the need for isolating the intermediate cyanoimidate. This procedure gives comparable yields but the crude product obtained is of lower purity and requires greater effort in purification compared to the first method described.

LC: 100%

MS: m/z 396.3 (M+1)

¹H-NMR (400 MHz, d⁶-DMSO) δ 7.52 (dt, 1H), 7.45 (dt, 1H), 7.21 (m, 2H), 4.97 (t, 1H), 4.55 (m, 1H), 4.38 (t, 2H), 3.76 (q, 2H), 2.88 (m, 2H), 2.61 (bt, 1H), 2.33 (m, 4H), 1.76-1.37 (m, 16H).

### 2-[2-Cyanoimino-3-(1-cyclooctyl-piperidinyl)-2,3-dihydro-benzoimidazol-1-yl]-acetamide (6)

The benzimidazole 5 (1.5 g, 4.27 mmol) was suspended in dry THF (50 mL) and cooled to 0° C. under nitrogen. Sodium hydride (95% dispersion in mineral oil, 118 mg, 4.69 mmol) was added and the mixture stirred for 30 min to give a clear solution. 2-Bromoacetamide (647 mg, 4.69 mmol) in THF (10 mL) was added and the mixture allowed to warm to room temperature then heated to 50° C. with stirring overnight. The cooled mixture was poured into water (500 mL), filtered and the solid washed with ethyl acetate (50 mL) to give the desired product 6 (1.23 g, 70%) as an off-white solid. TLC (SiO₂, EtOAc:MeOH:NH₃, 200:10:1) Rf=0.24 (UV or Dragendorff's reagent)

LC: 100%

MS: m/z 409.2 (M+1)

¹H-NMR (400 MHz, d⁶-DMSO) δ 7.75 (s, 1H), 7.52 (dd, 1H), 7.37 (s, 1H), 7.30 (dd, 1H), 7.20 (m, 2H), 4.96 (s, 2H), 4.55 (m, 1H), 3.33 (d, 2H), 2.88 (m, 2H), 2.62 (bt, 1H), 2.30 (m, 4H), 1.80-1.37 (m, 15H).

### Preparation of the Sulfamic Acid Salt of (6)

The free base (1.23 g, 3.01 mmol) was suspended in ethyl acetate (50 mL) and sulfamic acid (292 mg, 3.01 mmol) in water (3 mL) added with vigorous stirring. The mixture was stirred for 1 h then filtered and dried in vacuo to give the sulfamic acid salt of V112747 (1.4 g, 92%) as a pale yellow solid. TLC (SiO₂, EtOAc:MeOH:NH₃, 200:10:1) Rf=0.20 (UV or Dragendorff's reagent)

¹H-NMR (400 MHz, d⁶-DMSO) δ 7.80 (s, 1H), 7.70 (d, 1H, J=8 Hz), 7.42 (s, 1H), 7.38 (d, 1H, J=8 Hz), 7.27 (m, 2H), 5.5 (bs, 1H), 5.07 (s, 2H), 4.90 (bt, 1H), 3.45 (m, 2H), 3.25 (m, 1H), 2.72-2.79 (m, 2H), 1.96 (m, 4H), 1.45-1.80 (m, 12H).

### EXAMPLE 2

### Preparation of 1-(1-Cyclooctyl-piperidin-4-yl)-1,3-dihydro-benzoimidazol-2-ylidene-cyanamide and 2-[2-Cyanoimino-3-(1-cyclooctyl-piperidin-4-yl)-2,3-dihydro-benzoimidazol-1-yl]-acetamide

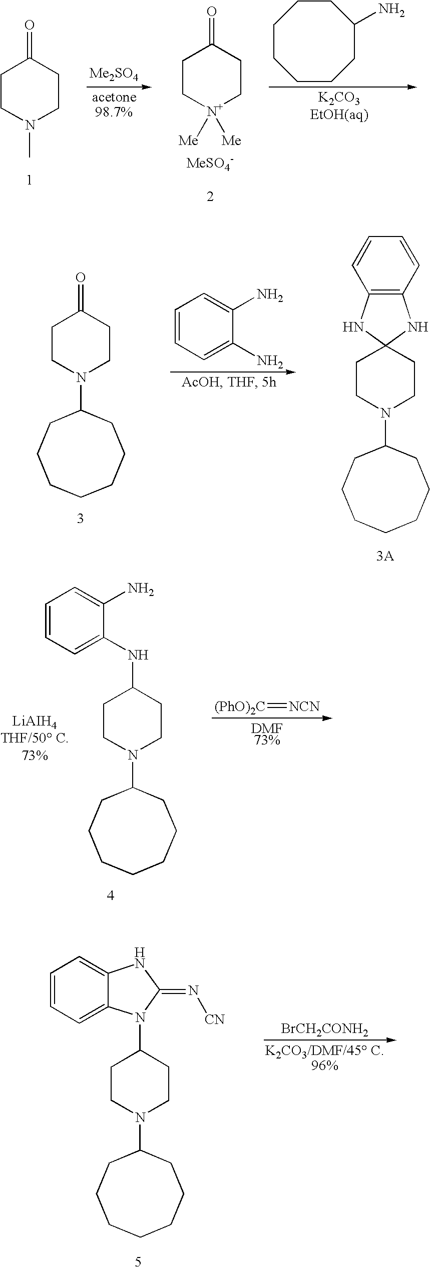

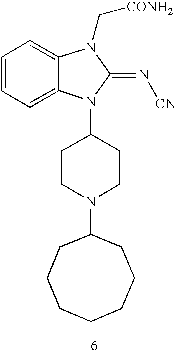

### 1,1-dimethyl-4-oxo-piperidinium methylsulfate (2)

N-Methyl-4-piperidone (107 mL, 0.945 mol) was dissolved in acetone (1,000 mL) and cooled to 0° C. with mechanical stirring. Dimethyl sulfate (90 mL) was added drop wise and the resulting heavy white precipitate stirred for 3 h. The mixture was filtered and the resulting solid washed with acetone (500 mL) to give a white solid, which was dried in vacuo at 40° C. to give the title compound (223 g, 98.7%).

¹H-NMR (400 MHz, CDCl₃) δ 3.74 (t, 4H, J=6 Hz), 3.37 (s, 3H), 3.26 (s, 6H), 2.71 (t, 4H, J=6 Hz).

### N-cyclooctyl-4-piperidone (3)

Cyclooctylamine (25.25 g, 198.5 mmol) was dissolved in ethanol (100 mL). Potassium carbonate (2.925 g, 20.895 mmol) in water (50 mL) was added and the mixture brought to reflux. 1,1-Dimethyl-4-oxo-piperidinium methylsulfate (50 g, 208.95 mmol) in ethanol:water (2:1, 300 mL) was added drop wise over 1.5 h using a pressure equalizing dropping funnel, and the resulting orange solution stirred for a further 1 h. The cooled solution was evaporated to dryness in vacuo and the residue partitioned between hexane (1,000 mL) and brine (1,000 mL). The organic phase was dried (MgSO₄) and the solvent evaporated to dryness in vacuo to leave an orange oil (30 g, 69%). ¹H NMR shows this material to be >90% pure with minor impurity peaks. The oil was vacuum distilled at 1 torr (114-116° C.) using a vigreaux column to give the title compound (25.1 g, 57%) as a colorless oil. TLC (SiO₂, ether) R.f.=0.60 detection Dragendorff's reagent.

¹H-NMR (400 MHz, CDCl₃) δ 2.78 (t, 4H, J=6 Hz), 2.75 (m, 1H), 2.43 (t, 4H, J=6 Hz), 1.80-1.43 (m, 14H).

The yield of this reaction may be improved by reducing the rate of addition of the piperidinium salt, in order to minimize the amount of reactive enone intermediate generated in situ. In addition some decomposition of the product was observed during distillation, which would be reduced by using a better vacuum (0.5 torr or lower) and therefore lowering the distillation temperature.

### N-(1-Cyclooctyl-piperidin-4-yl)-benzene-1,2-diamine (4)

o-Phenylenediamine (5.17 g, 47.8 mmol) and N-cyclooctyl-4-piperidone (10 g, 47.8 mmol) were dissolved in dry tetrahydrofuran (100 mL). Acetic acid (4.3 mL, 71.7 mmol) was added and the mixture stirred for 5 h. The orange solution was partitioned between ethyl acetate (400 mL) and 1M sodium carbonate (400 mL) and the organic phase separated. The aqueous phase was further extracted with ethyl acetate (100 mL) and the combined organics dried (MgSO₄) and the solvent evaporated to dryness in vacuo to leave an orange gum. This was dissolved in dry tetrahydrofuran (100 mL) and added via a pressure equalizing dropping funnel to a suspension of lithium aluminum hydride (3.6 g, 95.6 mmol) in dry tetrahydrofuran (300 mL) at 0° C. The mixture was then warmed to room temperature and then gently warmed to 50° C. over 6 h with stirring. The mixture was re-cooled to 0° C. and quenched by addition of 10% aqueous tetrahydrofuran (100 mL) followed by 1M sodium hydroxide (5 mL). The mixture was dried (MgSO₄) and filtered. The filtrate was evaporated to dryness in vacuo to leave an orange gum. This was dissolved in hexane:toluene (1:1) (ca 150 mL) and allowed to crystallize slowly at −10° C. (ice-acetone) with stirring. The mixture was filtered to give the title compound (7.5 g, 52%) as a white solid. The residue was chromatographed over flash silica (Merck SiO₂, 9385) eluting with ethyl acetate (3× column lengths) followed by ethyl acetate:methanol:ammonia (100:10:1) to give further title compound (3.0 g, 21%).

¹H-NMR (400 MHz, CDCl₃) δ 6.8 (t, 1H, J=12 Hz), 6.75 (d, 1H, J=12 Hz), 6.65 (m, 2H), 3.3 (bs, 1H), 3.2 (bs, 2H), 2.8 (bd, 2H), 2.60 (bt, 1H), 2.35 (t, 2H, J=10 Hz), 2.1 (d, 2H, J=10 Hz), 1.80-1.40 (m, 15H).

It is important that all the o-phenylenediamine is consumed in the first step, which may be achieved by the addition of a slight excess (1.05 equivalents) of N-cyclooctyl-4-piperidone. This makes the recrystallization of the diamine easier. Recrystallization may work better in cyclohexane. The ethyl acetate used for work up has been replaced with ether; it is likely that toluene could be used and azeotroped to dryness prior to addition of lithium aluminum hydride, thereby eliminating the need to completely remove the solvent.

### 1-(1-Cyclooctyl-piperidin-4-yl)-1,3-dihydro-benzimidazol-2-ylidene-cyanamide (5)

N-(1 Cyclooctyl-piperidin-4-yl)-benzene-1,2-diamine (10.0 g, 33.19 mmol) was added to a solution of diphenyl cyanocarbonimidate (8.69 g, 36.51 mmol) in dry N,N-dimethylformamide (150 mL) under argon, and the mixture stirred at room temperature for 1 h, then heated to 100° C. for 4 h. The solvent was removed in vacuo and the residue stirred with acetonitrile (200 mL) for 1 h with ice-water cooling, filtered and dried to give the title compound (8.4 g, 73%) as a buff colored solid.

MS: m/z 396.3 (M+1)

¹H-NMR (400 MHz, d⁶ DMSO) δ 7.52 (dt, 1H), 7.45 (dt, 1H), 7.21 (m, 2H), 4.97 (t, 1H), 4.55 (m, 1H), 4.38 (t, 2H), 3.76 (q, 2H), 2.88 (m, 2H), 2.61 (bt, 1H), 2.33 (m, 4H), 1.76-1.37 (m, 14H).

### 2-[2-Cyanoimino-3-(1-cyclooctyl-piperidin-4-yl)-2,3-dihydro-benzoimidazol-1-yl]-acetamide (6)

1-(1-Cyclooctyl-piperidin-4-yl)-1,3-dihydro-benzimidazol-2-ylidene-cyanamide (1.0 g, 2.84 mmol) was suspended in dry N,N-dimethylformamide (10 mL). Potassium carbonate (0.477 g, 3.41 mmol) was added followed by 2-bromoacetamide (0.392 g, 2.84 mmol) and the mixture stirred at room temperature for 1 h then at 45° C. for 2 h. The solvent was removed in vacuo and the residue diluted with water (25 mL) and filtered. The solid was washed with cold acetone (50 mL) to give the title compound (1.06 g, 91%). On standing the filtrate gave further precipitate, which was filtered off to give a second crop of title compound (0.06 g, 5%). TLC SiO₂ (EtOAc:MeOH:NH₃, 200:10:1) R.f.=0.24, detection UV, Dragendorff's reagent.

MS: m/z 409.2 (M+1)

¹H-NMR (400 MHz, d⁶ DMSO) δ 7.75 (s, 1H), 7.52 (dd, 1H), 7.37 (s, 1H), 7.30 (dd, 1H), 7.20 (m, 2H), 4.96 (s, 2H), 4.55 (m, 1H), 3.33 (d, 2H), 2.88 (m, 2H), 2.62 (bt, 1H), 2.30 (m, 4H), 1.80-1.37 (m, 14H).

### 2-[2-Cyanoimino-3-(1-cyclooctyl-piperidin-4-yl)-2,3-dihydro-benzoimidazol-1-yl]-acetamide sulfamate (6S)

The free base (9.2 g, 22.52 mmol) was dissolved in methanol (1,000 mL) with stirring and heating. Sulfamic acid (2.19 g, 22.52 mmol) in boiling water (10 mL) was added to form a clear solution. The mixture was concentrated to ca 200 mL in vacuo, and left to crystallize slowly in the freezer overnight. The mixture was filtered to give the title compound (10.5 g, 92%) as fluffy white needles. TLC (ethyl acetate:methanol:ammonia, 200:10:1) R.f.=0.20 detection UV, Dragendorff's reagent.

¹H-NMR (400 MHz, CDCl₃) δ 7.80 (s, 1H), 7.70 (d, 1H, J=8 Hz), 7.42 (s, 1H), 7.38 (d, 1H, J=8 Hz), 7.27 (m, 2H), 5.5 (bs, 1H), 5.07 (s, 2H), 4.90 (bt, 1H), 3.45 (m, 2H), 3.25 (m, 1H), 2.72 9m, 2H), 1.96 (m, 4H), 1.45-1.80 (m, 12H).

### EXAMPLE 3

### Preparation of N-Cyclooctyl-piperidone

To a solution of 4-bromomethylbenzoic acid (100 gram, 97%, 0.45 mol) in 3 L of acetone at room temperature was added a solution of N-methyl piperidone (44 gram, 0.45 mol) in 50 ml acetone with stir. After the addition, the volume was adjusted to 3.5 L with additional acetone. The clear light yellow solution was stirred at room temperature. Within 20 minutes the solution become cloudy. Within 1 hour some white precipitate was observed and stirring was stopped and the mixture was left un-stirred at room temperature for 16 hours. The white precipitates were collected via filtration with Buchner funnel. The solid was further washed with acetone and then hexane. The white solid was then dried in a glass dish under mild heat with stir for 1 hour resulting free-flowing white solid. The mother liquid gave additional product and was dried as before. The weight was recorded until it does not change and stabilized.

First batch: 129 gram; second batch: 10 gram; third batch: 3 gram. Total yield: 91%

Conversion to Title Compound

To a 2 L RB-flask was added quaternary salt (20.2 gram, 58.4 mmol) followed by addition of 560 ml of distilled water. To the mixture was added a solution of cyclooctyl amine (7.3553 gram, 97%, 56.2 mmol) in 20 ml of ethanol at room temperature, additional 260 ml of ethanol was added and the resulting mixture was stirred at room temperature for 10 minutes to generate a clear solution. To this solution was then added NaOH (2.699 gram, 97%, 65.4 mmol) and the RB flask was equipped with a water-cooled condenser and heated in 70-80° C. oil bath for 4 hours. The reaction mixture was concentrated on rotary evaporator with 40° C. water bath to 250 ml (<⅓ of the original volume). The aqueous was extracted with 400 ml of hexane. The hexane portion was washed with 150 ml of saturated NaHCO3 aq followed by 150 ml of brine. LCMS of the combined aqueous wash does not show product peak, thus the wash was not combined with reaction mixture. Also the LCMS of the reaction mixture shows that after the first hexane extraction, very small product peak remains. Nevertheless, two more extractions with same volume of hexane was applied followed by 150 ml brine wash. The combined hexane portion was dried with MgSO4, filtered and concentrated on the rotary evaporator at 40° C. water bath temperature to give 8.0069 gram (68%) pure compound.

1. A process for synthesizing a compound of formula (V): 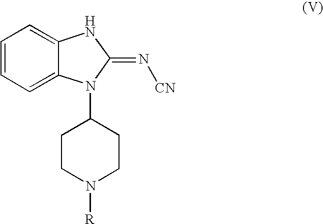
comprising reacting a compound of formula (IV): 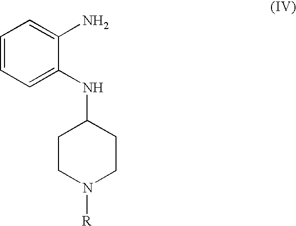
with (A)(A₁)-cyanocarbonimidate to form a compound of formula (V);
wherein A and A₁ are independently selected from methyl, ethyl propyl, phenyl and benzyl; and wherein,
R is Z-R1, wherein
Z is selected from the group consisting of a bond, straight or branched C₁₋₆ alkylene, —NH—, —CH₂O—, —CH₂NH—, —CH₂N(CH₃)—, —NHCH₂—, —CH₂CONH—, —NHCH₂CO—, —CH₂CO—, —COCH₂—, —CH₂COCH₂—, —CH(CH₃)—, —CH═, —O— and —HC═CH—, wherein the carbon and/or nitrogen atoms are unsubstituted or substituted with one or more lower alkyl, hydroxy, halo or alkoxy group;
R₁ is selected from the group consisting of hydrogen, C₁₋₁₀ alkyl, C₃₋₁₂cycloalkyl, C₂₋₁₀alkenyl, amino, C₁₋₁₀alkylamino-, C₃₋₁₂cycloalkylamino-, —COOV₁, —C₁₋₄COOV₁, cyano, cyanoC₁₋₁₀alkyl-, cyanoC₃₋₁₀cycloalkyl-, NH₂SO₂—, NH₂SO₂C₁₋₄alkyl-, NH₂SOC₁₋₄alkyl-, aminocarbonyl-, C₁₋₄alkylaminocarbonyl-, diC₁₋₄alkylaminocarbonyl-, benzyl, C₃₋₁₂ cycloalkenyl-, a monocyclic, bicyclic or tricyclic aryl or heteroaryl ring, a hetero-monocyclic ring, a hetero-bicyclic ring system, and a spiro ring system of the formula (XI): 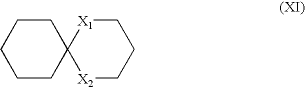
wherein X₁ and X₂ are independently selected from the group consisting of NH, O, S and CH₂; and wherein said alkyl, cycloalkyl, alkenyl, C₁₋₁₀alkylamino-, C₃₋₁₂cycloalkylamino-, or benzyl of R₁ is optionally substituted with 1-3 substituents selected from the group consisting of halogen, hydroxy, C₁₋₁₀ alkyl, C₁₋₁₀ alkoxy, nitro, trifluoromethyl-, cyano, —COOV₁, —C₁₋₄COOV₁, cyanoC₁₋₁₀alkyl-, —C₁₋₅(═O)W₁, —C₁₋₅NHS(═O)₂W₁, —C₁₋₅NHS(═O)W₁, a 5-membered heteroaromaticC₀₋₄alkyl-, phenyl, benzyl, benzyloxy, said phenyl, benzyl, and benzyloxy optionally being substituted with 1-3 substituents selected from the group consisting of halogen, C₁₋₁₀ alkyl-, C₁₋₁₀ alkoxy-, and cyano; and wherein said C₃₋₁₂ cycloalkyl, C₃₋₁₂ cycloalkenyl, monocyclic, bicyclic or tricyclic aryl, heteroaryl ring, hetero-monocyclic ring, hetero-bicyclic ring system, or spiro ring system of the formula (XI) is optionally substituted with 1-3 substituents selected from the group consisting of halogen, C₁₋₁₀ alkyl, C₁₋₁₀ alkoxy, nitro, trifluoromethyl-, phenyl, benzyl, phenyloxy and benzyloxy, wherein said phenyl, benzyl, phenyloxy or benzyloxy is optionally substituted with 1-3 substituents selected from the group consisting of halogen, C₁₋₁₀ alkyl, C₁₋₁₀ alkoxy, and cyano;
wherein V₁ is independently selected from H, C₁₋₆ alkyl, C₃₋₆ cycloalkyl, benzyl and phenyl; and
wherein W₁ is hydrogen, C₁₋₁₀ alkyl, C₃₋₁₂ cycloalkyl, C₁₋₁₀ alkoxy, C₃₋₁₂ cycloalkoxy, —CH₂OH, amino, C₁₋₄alkylamino-, or diC₁₋₄alkylamino-.

2. The process of claim 1, wherein the compound of formula (IV) is formed by subjecting a compound of formula (III): 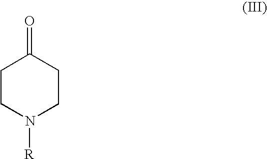
to reductive amination with 1,2-phenylenediamine, an acid and a reducing agent to form a compound of formula (IV).

3. The process of claim 1, wherein the compound of formula (IV) is formed by subjecting a compound of formula (III): 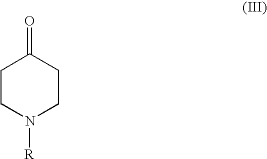
to amination with 1,2-phenylenediamine and an acid to form a compound of formula (IIIA): 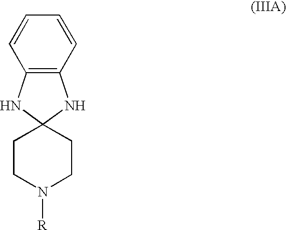
and reducing the compound of (IIIA) with a reducing agent to form a compound of formula (IV).

4. The process of claim 2, wherein the compound of formula (III) is formed by reacting a compound of formula (II): 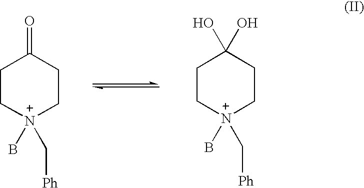
with R-amine to form a compound of formula (III);
wherein B is selected from the group consisting of methyl, ethyl and propyl.

5. The process of claim 2, wherein the compound of formula (III) is formed by reacting a compound of formula (IIA): 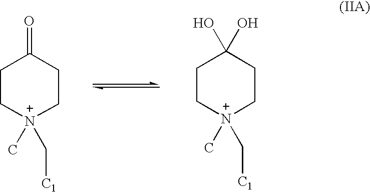
with R-amine to form a compound of formula III;
wherein C and C₁ are independently selected from the group consisting of methyl, ethyl and propyl.

6. The process of claim 4, wherein the compound of formula (II) is formed by reacting a compound of formula (I): 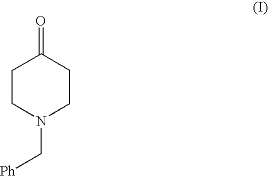
with an C₁₋₃alkyl-halogen to form a compound of formula (II).

7. The process of claim 4, wherein the compound of formula (II) is formed by reacting a compound of formula (IA): 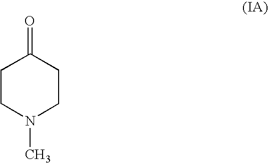
with a benzyl-halogen to form a compound of formula II.

8. The process of claim 4, wherein the compound of formula (IIA) is formed by reacting a compound of formula (IA): 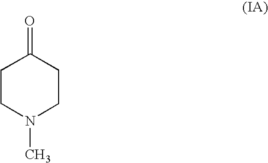
with (C)(C₁)sulphate to form a compound of formula (IIA).

9. The process of claim 1, further comprising reacting a compound of formula (V) with a D-halogen to form a compound of formula (VI): 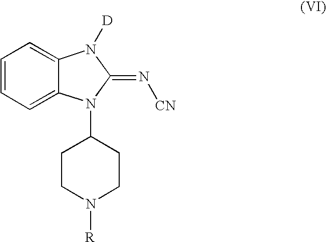
wherein D is selected from the group consisting of C₁₋₁₀ alkyl, C₃₋₁₂ cycloalkyl, C₃₋₁₂ cycloalkylC₁₋₄alkyl-, C₁₋₁₀ alkoxy, C₃₋₁₂ cycloalkoxy-, C₁₋₁₀ alkyl substituted with 1-3 halogen, C₃₋₁₂ cycloalkyl substituted with 1-3 halogen, C₃₋₁₂ cycloalkylC₁₋₄alkyl-substituted with 1-3 halogen, C₁₋₁₀ alkoxy substituted with 1-3 halogen, C₃₋₁₂ cycloalkoxy-substituted with 1-3 halogen, —COOV₁, —C₁₋₄COOV₁, —CH₂OH, —SO₂N(V₁)₂, hydroxyC₁₋₁₀alkyl-, hydroxyC₃₋₁₀cycloalkyl-, cyanoC₁₋₁₀alkyl-, cyanoC₃₋₁₀cycloalkyl-, —CON(V₁)₂, NH₂SO₂C₁₋₄alkyl-, NH₂SOC₁₋₄alkyl-, sulfonylaminoC₁₋₁₀alkyl-, diaminoalkyl-, -sulfonylC₁₋₄alkyl, a 6-membered heterocyclic ring, a 6-membered heteroaromatic ring, a 6-membered heterocyclicC₁₋₄alkyl-, a 6-membered heteroaromaticC₁₋₄alkyl-, a 6-membered aromatic ring, a 6-membered aromaticC₁₋₄ alkyl-, a 5-membered heterocyclic ring optionally substituted with an oxo or thio, a 5-membered heteroaromatic ring, a 5-membered heterocyclicC₁₋₄alkyl-optionally substituted with an oxo or thio, a 5-membered heteroaromaticC₁₋₄alkyl-, —C₁₋₅(═O)W₁, —C₁₋₅(═NH)W₁, —C₁₋₅NHC(═O)W₁, —C₁₋₅NHS(═O)₂W₁, —C₁₋₅NHS(═O)W₁, and a 5-membered heteroaromatic ring optionally substituted with 1-3 lower alkyl
wherein V₁ is independently selected from H, C₁₋₆ alkyl, C₃₋₆ cycloalkyl, benzyl and phenyl; and
wherein W₁ is hydrogen, C₁₋₁₀ alkyl, C₃₋₁₂ cycloalkyl, C₁₋₁₀ alkoxy, C₃₋₁₂ cycloalkoxy, —CH₂OH, amino, C₁₋₄alkylamino-, or diC₁₋₄alkylamino-; and
wherein each V₁ and W₁ is the same or different.

10. The process of claim 1, wherein R₁ is selected from the group consisting of C₁₋₁₀alkyl and C₃₋₁₂cycloalkyl.

11. The process of claim 1, wherein R is cyclooctyl.

12. The process of claim 1, wherein A and A₁ are both phenyl.

13. The process of claim 1, wherein the reaction is performed in a solvent.

14. The process of claim 13, wherein the solvent is selected from acetonitrile, dimethylformamide, or a mixture thereof.

15. The process of claim 1, wherein the reaction is performed at a temperature of about 50° C. to about 125° C. or about 75° C. to about 125° C. or about 100° C.

16. The process of claim 15, wherein a portion of the reaction is performed under ambient temperature.

17. The process of claim 1, comprising isolating an intermediate cyanoimidate.

18. The process of claim 17, comprising preparing the compound of formula (V) in a one pot reaction in acetonitrile, dimethylformamide, or a mixture thereof.

19. The process of claim 2, wherein the reductive amination is performed in a suitable solvent.

20. The process of claim 19, wherein the solvent is dichloroethane, tetrahydrofuran or a mixture thereof.

21-76. (canceled)
